(19) United States
(12) Patent Application Publication
(10) Pub. No.: US 2026/0199096 A1
(43) Pub. Date: Jul. 16, 2026

(54) A DUAL MOBILITY PROTHESIS FOR IMPLANTATION INTO THE ACETABULUM

(71) Applicant: MatOrtho Liimited, Leatherhead, Surrey (GB)

(72) Inventors: Simon Nicholas Collins, Leatherhead, Surrey (GB); Michael Antony Tuke, Leatherhead, Surrey (GB); Danielle Simone De Villiers, Leatherhead, Surrey (GB)

(21) Appl. No.: 19/136,077

(22) PCT Filed: Dec. 8, 2023
(86) PCT No.: PCT/GB2023/053162

§ 371 Date: Jun. 5, 2025

(30) Foreign Application Priority Data
Dec. 15, 2022 (GB) GB 2218957.5

(51) Int. Cl. A61F 2/36; A61F 2/30; A61F 2/34

(52) U.S. Cl. CPC A61F 2/3601; A61F 2002/3065; A61F 2002/30654; A61F 2002/3483

(57) ABSTRACT

A dual mobility prosthesis (10) for implantation into acetabulum, dual mobility prosthesis (10) comprising a monobloc ceramic acetabular cup (12) having an internal concave surface forming a first articulation surface (18); a polyethylene articulation element (14) having a convex outer surface forming a second articulation surface (28), and a concave internal surface forming a third articulation surface (30), the second articulation surface (28) being rotatably engageable with the first articulation surface (18) of the monobloc ceramic acetabular cup (12); a ceramic femoral ball head (16) having a convex outer surface forming a fourth articulation surface (40) directly engageable against the third articulation surface (30) of the polyethylene articulation element (14), the ceramic femoral ball head (16) being attachable to a femoral stem.

The present invention relates to a dual mobility prosthesis for implantation into the acetabulum of a patient, preferably but not necessarily exclusively used in total hip replacement surgery applications, a dual mobility prosthesis system, a method of assembly of a dual mobility prosthesis using the system, and a total hip implant system.

Total hip replacement (THR) is a surgical procedure in which the hip joint is replaced by a prosthesis. THR commonly involves replacing the acetabulum and the femoral head to provide a patient with a replacement artificial ball-and-socket joint. THR is a commonly performed orthopaedic operation.

A risk with THR is dislocation of the ball from the socket. To reduce this risk, dual mobility components are often used. A dual mobility component includes an acetabular cup for placement in the acetabulum of a patient. The acetabular cup is typically formed from a metal-containing material, for example, a Titanium or Cobalt Chromium alloy. The dual mobility component also includes a liner that engages with the acetabular cup via friction-fit engagement. The liner is stationary relative to the acetabular cup. The dual mobility component further comprises a head portion. The head portion is comprised of an outer ball head and an inner femoral ball head. The outer ball head is commonly made from polyethylene. The inner femoral ball head is commonly at least partly formed from metal. The outer ball head is located rotatably inside the liner, whilst the inner femoral ball head is located rotatably inside the outer ball head. The combination of the two heads allows for motion in different arcs simultaneously, in other words, it provides dual mobility. The head portion couples with a hip stem. The hip stem may be made of metals such as titanium, cobalt-based alloys, or high-nitrogen steel.

The use of a dual mobility component often requires assembly of an outer metal acetabular cup shell and a metal or ceramic liner at implantation. This introduces the potential for bodily fluid to enter the space between the liner and cup. The bodily fluid can then act as an electrolytic medium facilitating ion exchange between the acetabular cup and the liner due to relative energy differentials. In time this causes surface level ionization of the metal acetabular cup and/or metal liner. For example, in cases where the acetabular cup is manufactured from Cobalt Chromium, Cobalt ions may break free and enter the bloodstream. Furthermore, due to micro-motion abrasion in use, the use of a liner can result in particles of the metal acetabular cup entering the body. Friction between the artificial femoral ball head and the liner, if either are made at least in part of metal, may also result in metal entering the body. The metal ions and particles may be toxic and carcinogenic.

The potential for malalignment of a tapered portion of the liner and cup can also occur allowing for movement and dissociation of the liner. While pre-assembly of the liner in the cup is an option to reduce the risk of misalignment, the dissociation of the liner still occurs clinically sometimes as a result of impingement due to extreme ranges of motion or sub-optimal cup orientation. This introduces the potential for wear at the liner cup interface which can lead to metallosis. Liner misalignment can also result in early fracture of ceramic liners when these are used.

The present invention therefore seeks to provide a solution which obviates or overcomes the above-mentioned deficiencies, by providing a dual mobility prosthesis that does not result in the dissolution of metal ions into the body.

According to a first aspect of the invention, there is provided a dual mobility prosthesis for implantation into acetabulum, the dual mobility prosthesis comprising: a monobloc ceramic acetabular cup having an internal concave surface forming a first articulation surface; a polyethylene articulation element having a convex outer surface forming a second articulation surface, and a concave internal surface forming a third articulation surface, the second articulation surface being rotatably engageable with the first articulation surface of the monobloc ceramic acetabular cup; a ceramic femoral ball head having a convex outer surface forming a fourth articulation surface directly engageable against the third articulation surface of the polyethylene articulation element, the ceramic femoral ball head being attachable to a femoral stem; wherein the first articulation surface is dimensioned to contact the second articulation surface and covers less than a hemisphere to non-captively receive the polyethylene articulation element.

Ceramic has excellent biocompatibility, high surface hardness and wear resistance. The use of only ceramic and polyethylene within the dual mobility prosthesis prevents toxic metal ions entering the bloodstream as no metal components are used. Therefore, carcinogenic and genotoxic effects that commonly arise when metal components are used, are prevented. The use of a polyethylene articulation elements is common in THR however less wear is produced against ceramic compared to metal interfaces. The provision of the four articulation surfaces allows for articulation of the polyethylene articulation element relative to both the monobloc ceramic acetabular cup and the ceramic femoral ball head, as well as allowing for independent articulation of the ceramic femoral ball head relative to both the monobloc ceramic acetabular cup and polyethylene articulation element. The intermediate polyethylene articulation element separates the ceramic components and acts as an articulating bearing providing range of motion benefits.

The first articulation surface contacting less than a hemisphere of the second articulation surface allows for the articulation characteristics of the polyethylene articulation element to be enhanced. A femoral hip stem attached to the ceramic femoral ball head is less likely to impinge the monobloc ceramic acetabular cup. The first articulation surface may have an active arc of less than or equal to 180 degrees, so the second articulation surface is not restricted, but there is sufficient contact between the first and second articulation surfaces. As a result of the above, the second articulation surface has an increased degree of rotation with a decreased risk of stem impingement.

Preferably, the third articulation surface may be dimensioned to contact the fourth articulation surface and may cover more than a hemisphere to captively receive the ceramic femoral ball head.

The third articulation surface covering more than a hemisphere of the fourth articulation surface ensures the ceramic femoral ball head is captively received by the polyethylene articulation element. The third articulation surface preferably covers more than 180 degrees of the fourth articulation surface.

Preferably, the ceramic femoral ball head may connect to the polyethylene articulation element via a push-fit mechanism.

A push-fit mechanism is a simple and easy mechanism by which a surgeon can ensure the ceramic femoral ball head is enclosed within the polyethylene articulation element. A push-fit mechanism is also a fast and secure mechanism of attachment, simplifying the installation of the prosthetic into a patient.

Optionally, the polyethylene articulation element may be made of a conventional Ultra High Molecular Weight Polyethylene, and/or crosslinked polyethylene and/or a vitamin-E crosslinked polyethylene.

Polyethylene is commonly used within dual mobility prostheses; however, polyethylene is worn away over time and so wear particles are generated. The conventional Ultra High Molecular Weight Polyethylene typically has a radiation dose of approximately 25 to 35 kilograys and has low cross-linking. Highly crosslinked polyethylene has a higher wear resistance than conventional polyethylene and thus less wear particles result. Therefore, as crosslinked polyethylene has excellent long-term durability, and less wear particles it is suited for placement between the monobloc ceramic acetabular cup and the ceramic femoral ball head. The inclusion of vitamin-E in crosslinked polyethylene further provides ongoing wear resistance, whilst remaining biocompatible, by reducing free radicals that lead to polyethylene oxidation during use.

Advantageously, the polyethylene articulation element may directly engage with the monobloc ceramic acetabular cup without a separate liner.

In the present invention, the monobloc ceramic acetabular cup provides an outer surface for bony ingrowth as well as the first articulation surface. Negating the need for a liner may also simplify the implantation of the dual mobility prosthesis. Additionally, the absence of a liner may increase the articulation surface available so that the articulation extent of the polyethylene articulation element is increased. Eliminating a separate liner may decrease the wall thickness of the total acetabular cup, as conventionally the wall thickness of the total acetabular cup includes the acetabular cup and liner. Having an overall decreased wall thickness means as little bone as possible may be removed from the from the implant area to allow for a future revision. This also aids to preserve the natural physiology of the patient. An overall decreased wall thickness may also allow a wider selection of femoral head sizes to be used. A more thinly-walled cup allows for larger diameter polyethylene articulation element to be used, and therefore a larger range of motion.

Preferably, an equatorial centre point of the first articulation surface may be offset from an equatorial centre of the third articulation surface.

The offset equatorial centre point reduces the risk of stem impingement in use. As such, the offset equatorial centre point also maximises the possible articulation extent of both the polyethylene articulation element and the ceramic femoral ball head. An artificial femoral stem colliding with the edges of the monobloc ceramic acetabular cup may result in potential ceramic edge chipping and/or looseness, and even dislocation of a femoral ball head.

Preferably, the ceramic acetabular cup may have a convex outer contact surface engagable with an acetabulum of a patient. Optionally, the convex outer contact surface may flare at or adjacent to a rim of the ceramic acetabular cup.

A convex outer contact surface engagable with an acetabulum of a patient allows the ceramic acetabular cup to be placed in a patient, by a surgeon, with relative ease. Flaring of the convex outer contact surface may provide a gripping surface on the ceramic acetabular cup, and/or may improve robustness of the cup to reduce manufacturing defects. The flaring may also help with introduction or removal of the cup and manufacture of the ceramic cup.

Preferably, the second articulation surface has an articulation angle of at least 10 degrees greater than the articulation angle of the first articulation surface. Preferably, the second articulation surface may have an articulation angle of up to 50 degrees greater than the articulation angle of the first articulation surface. Most preferably, the articulation angle of the second articulation surface is 43 degrees greater than the articulation angle of the first articulation surface.

The articulation angle of the second articulation surface being at least 10 degrees greater than the articulation angle of the first articulation surface reduces the risk of stem impingement. The difference in articulation angle also allows for increased articulation characteristics of the second articulation surface.

Preferably, the fourth articulation surface may have an articulation angle of at least 10 degrees greater than the third articulation surface. More preferably, the fourth articulation surface may have an articulation angle of up to 60 degrees greater than the third articulation surface.

The articulation angle of the fourth articulation surface being at least 10 degrees greater than the articulation angle of the third articulation surface reduces the risk of stem impingement.

Optionally, the concave internal surface of the polyethylene articulation element may have a tapered portion.

The tapered portion reduces risk of looseness, and even dislocation of the femoral ball head upon impingement of the stem on the polyethylene articulation element. The taper provides a flat surface against which the stem can abut. The flat surface, as opposed to a corner or conventional rim surface reduces the point force on the stem. The tapering of the polyethylene articulation element may act as a cushioning element. Ceramic has low fracture toughness and bad impact-resistance, as such the taper helps to alleviate these issues by preventing a stem in connection with the ceramic femoral ball head from contacting the monobloc ceramic acetabular cup, as the tapering of the polyethylene articulation element guides the stem at an angle away from the monobloc ceramic acetabular cup. Contact between the stem and the monobloc ceramic acetabular cup would potentially result in damage of one or both.

Preferably, the tapered portion is a frusto-conical portion. Preferably, the frusto-conical portion of has a cone angle of 40 and 90 degrees.

Frusto-conical is a simplistic shape and easy to manufacture. The frusto-conical shape provides a flat-wall portion against which the femoral stem may abut. The flat-wall portion allows for the force from the femoral stem on the polyethylene articulation element to be spread. This in turn reduces risk of femoral stem impingement damage. An angle of 40 to 90 degrees helps to angle the femoral stem away from the monobloc ceramic acetabular cup to prevent stem impingement on the monobloc ceramic acetabular cup.

Preferably, the tapered portion extends from or is adjacent to a rim of the polyethylene articulation element.

The tapered portion extending from the rim may also help with insertion or removal, as the tapered portion may act as a gripping portion for the surgeon.

Preferably, a wall thickness of the ceramic acetabular cup is non-uniform, the thickness decreasing towards a rim of the ceramic acetabular cup.

Having a thin-walled ceramic acetabular cup potentially allows for a larger diameter of polyethylene articulation element and therefore permits a larger range of motion. Having a thicker central portion of the ceramic acetabular may improve wear-resistance.

Preferably, a wall thickness of the polyethylene articulation element is non-uniform, the thickness increasing towards a rim of the polyethylene articulation element.

The provision of a thicker rim may improve wear-resistance.

According to a second aspect of the invention, there is provided a dual mobility prosthesis system comprising: a plurality of monobloc ceramic acetabular cups, each monobloc ceramic acetabular cup having a cup articulation surface; a plurality of polyethylene articulation elements, each polyethylene articulation element having an inner articulation surface, and an outer articulation surface, the outer articulation surface being rotatably engageable with the cup articulation surface of the acetabular cup; and a plurality of ceramic femoral ball heads, each ceramic femoral ball head having a head articulation surface directly engageable against the inner articulation surface of the polyethylene articulation element, the ceramic femoral ball head being attachable to a femoral stem; wherein the cup articulation surface of each monobloc ceramic acetabular cup is dimensioned to contact the inner articulation surface of a selected polyethylene articulation element and covers less than a hemisphere to non-captively receive the selected polyethylene articulation element.

The concept of the present invention is to provide a dual mobility prosthesis system without a separate liner and without metal components. The provision of a plurality of monobloc ceramic acetabular cups, a plurality of polyethylene articulation elements and a plurality of ceramic femoral ball heads allows a surgeon to choose a suitable arrangement of monobloc ceramic acetabular cup, polyethylene articulation element and ceramic femoral ball head. Thus, the dual mobility prosthesis may be tailored to a patient's needs.

Preferably, at least one of the plurality of monobloc ceramic acetabular cups may be of a different dimension and/or shape to at least one other monobloc ceramic acetabular cup, and/or at least one of the plurality of polyethylene articulation elements is of a different dimension and/or shape to at least one other polyethylene articulation element, and/or at least one of the plurality of ceramic femoral ball head is of a different dimension and/or shape to at least one other ceramic femoral ball head.

The inclusion of different dimensioned and/or shaped monobloc ceramic acetabular cups, polyethylene articulation elements and ceramic femoral ball heads enhances the utility of the system as a whole. It allows the surgeon or medical professional to select a dual mobility prosthesis system that is suited towards the patient's needs. A bespoke dual mobility prosthesis can be created for each patient.

According to a third aspect of the invention, there is provided a method of ex vivo assembly of a dual mobility prosthesis, the method comprising the steps of: a] providing a dual mobility prosthesis system in accordance with the second aspect of the invention; b] selecting a monobloc ceramic acetabular cup having a convex outer contact surface corresponding to an acetabulum of a patient; and c] selecting a head assembly from a plurality of head assemblies, the head assembly comprising a polyethylene articulation element and a ceramic femoral ball head, the head assembly engageable with the monobloc ceramic acetabular cup and suitable for engagement with a femoral stem, the selection being based on an articulation characteristic required by a patient.

The present invention is particularly suited to assembling customised dual mobility acetabular prostheses for use in patients who have different mobility needs. The system does not require a separate liner and none of the components are made of metal. As such, the risk of dissolution of metal ions into the body is nil. The method of assembly allows a surgeon to retain as much of a patient's natural acetabulum as possible, by customising the monobloc ceramic acetabular cup used, the polyethylene articulation element used, and the ceramic femoral ball head used. The choice of components allows a surgeon to decide the stability, mobility and rotational extent of the dual mobility prosthesis in relation to a specific patient. In some cases, it may be preferable to have increased articulation of the polyethylene articulation element, and so thin-walled monobloc ceramic acetabular cup may be utilised to provide a larger rotational arc. In other cases, it may be preferable to have a thicker-walled monobloc ceramic acetabular cup for stability, although this may restrict the articulation extent of the polyethylene articulation element. The system allows a user assembling the dual mobility prosthesis to customise it to each individual patient according to the necessary specifications of that patient.

Preferably, selecting a head assembly comprises the steps of: a] selecting a polyethylene articulation element, the polyethylene articulation element being determined by a degree of rotation required by a patient; and b] selecting a ceramic femoral ball head engageable with the polyethylene articulation element.

A plurality of monobloc ceramic acetabular cups may be provided. As such, monobloc ceramic acetabular cups with different may be provided having different wall thicknesses. A polyethylene articulation element which provides the right balance of articulation and thickness may then be selected for use. Selecting the ceramic femoral ball head may be beneficial as it ensures the patient contacting component monobloc ceramic acetabular cup and rotational requirements are compatible with the patient foremost.

Preferably, the ceramic femoral ball head may connect to the polyethylene articulation element via a push-fit mechanism.

A push-fit mechanism is a simple mechanism to connect the polyethylene articulation element and ceramic femoral ball head. A push-fit mechanism is a secure mechanism of attachment, and as such simplifies the installation of the prosthetic into a patient.

According to a fourth aspect of the invention, there is provided a total hip implant system comprising: a dual mobility prosthesis in accordance with the first aspect of the invention; and a femoral stem implant attachable to a ceramic femoral ball head, wherein the femoral stem implant is locatable in a femur of a patient in use.

Providing a whole hip implant system allows for a surgeon or medical personnel to have all of the components necessary at hand. This also ensure that the femoral stem implant is compatible with the dual mobility prosthesis.

In the state of the art, dual mobility acetabular prostheses utilise metal components and liners. However, this can lead to the dissolution of potentially carcinogenic and/or toxic metal ions into the body. Advantageously, the present invention provides a dual mobility prosthesis that does not utilise metal components, and as such does not result in the dissolution of metal ions. The dual mobility prosthesis instead utilises ceramic for the acetabular cup and femoral ball head. Ceramic has excellent histocompatibility, does not release potentially toxic metal ions, has a high surface hardness and wear resistance. Therefore, the use of only ceramic and polyethylene within the dual mobility prosthesis eliminates the likelihood of toxic metal ions entering the bloodstream from the dual mobility prosthesis. The present invention therefore reduces carcinogenic and genotoxic effects associated with state of the art dual mobility prosthesis.

According to a further aspect of the invention, there is provided a dual mobility prosthesis for implantation into acetabulum, the dual mobility prosthesis comprising: a monobloc ceramic acetabular cup having an internal concave surface forming a first articulation surface; a polyethylene articulation element having a convex outer surface forming a second articulation surface, and a concave internal surface forming a third articulation surface, the second articulation surface being rotatably engageable with the first articulation surface of the monobloc ceramic acetabular cup; a ceramic femoral ball head having a convex outer surface forming a fourth articulation surface directly engageable against the third articulation surface of the polyethylene articulation element, the ceramic femoral ball head being attachable to a femoral stem.

The invention will now be more particularly described, by way of example only, with reference to the accompanying drawings, in which:

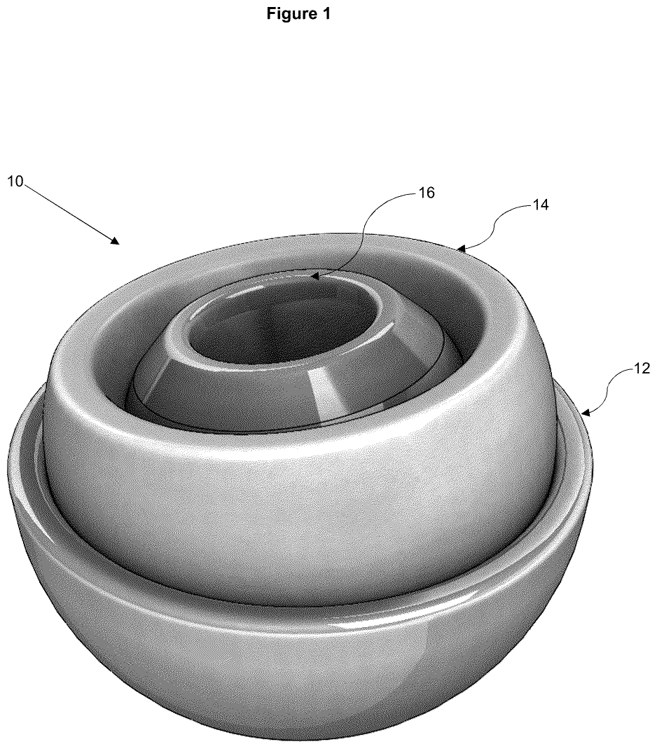

FIG. 1 shows a perspective side view of an embodiment of a dual mobility prosthesis in accordance with the first aspect of the invention;

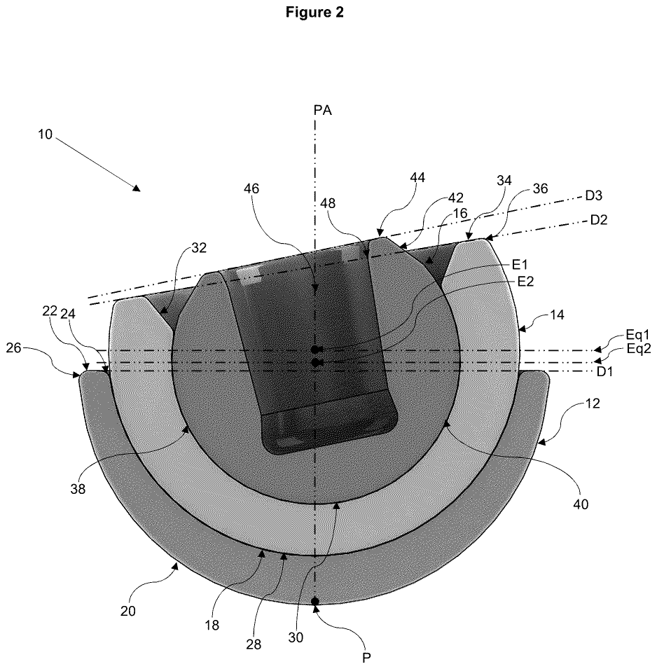

FIG. 2 shows a cross-section view of the dual mobility prosthesis of FIG. 1;

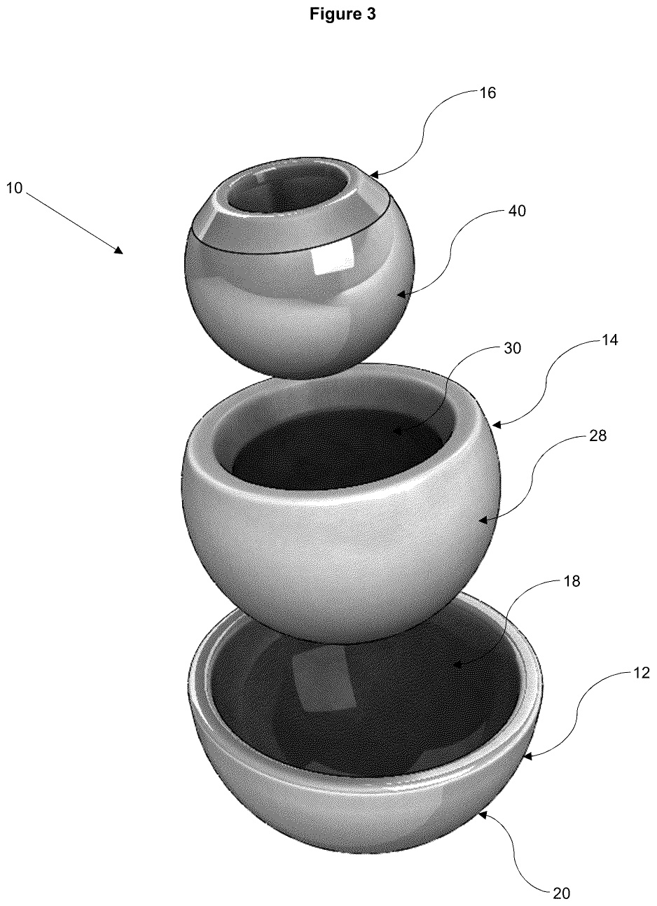

FIG. 3 shows an exploded perspective view of the dual mobility prosthesis of FIG. 1;

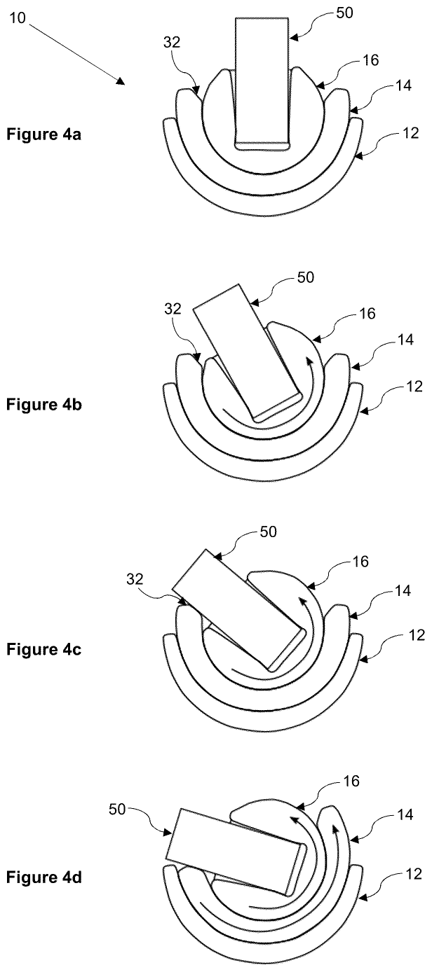

FIG. 4a shows a cross-sectional side view of a dual mobility prosthesis in accordance with the first aspect of the invention in use, with an associated femoral stem inserted into a ceramic femoral ball head of the dual mobility prosthesis;

FIG. 4b shows the dual mobility prosthesis of FIG. 4a, wherein the ceramic femoral ball head has rotated anti-clockwise relative to a polyethylene articulation element;

FIG. 4c shows the dual mobility prosthesis of FIG. 4b, wherein the associated femoral stem abuts a rim of the polyethylene articulation element;

FIG. 4d shows the dual mobility prosthesis of FIG. 4c, wherein the polyethylene articulation element has rotated due to contact with the associated femoral stem; and

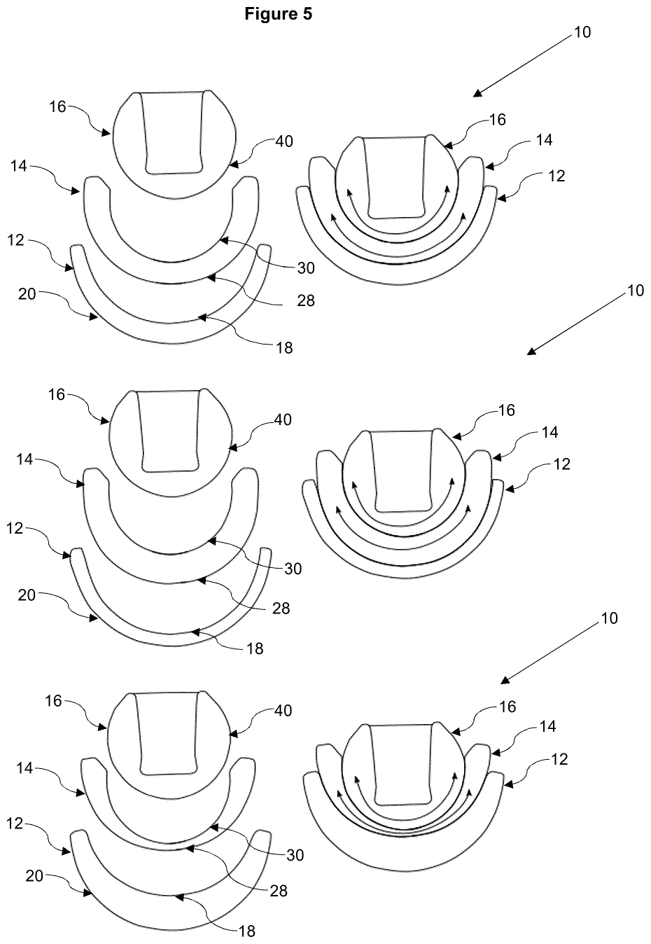

FIG. 5 shows a cross-sectional side view of an embodiment of a dual mobility prosthesis system in accordance with the second aspect of the invention.

Referring to FIG. 1, there is indicated a dual mobility prosthesis referenced globally at 10. The dual mobility prosthesis 10 comprises a monobloc ceramic acetabular cup 12, a polyethylene articulation element 14 and a ceramic femoral ball head 16. The ceramic femoral ball head 16 is positioned inside the polyethylene articulation element 14 which is in turn positioned inside the monobloc ceramic acetabular cup 12. The ceramic femoral ball head 16 can therefore be considered to form a head assembly with the polyethylene articulation element 14, with the polyethylene articulation element 14 acting as an intermediary bearing surface between the ceramic femoral ball head 16 and monobloc ceramic acetabular cup 12.

As best shown in FIG. 2, the monobloc ceramic acetabular cup 12 is dimensioned to fit an acetabulum of a patient. The monobloc ceramic acetabular cup 12 is formed solely of ceramic material. The ceramic material may be Aluminium Oxide based. For example, the ceramic material may include a percentage of Aluminium Oxide in a range of 15% to 85%. The ceramic material may contain Zirconium Oxide. For example, the ceramic material may include a percentage of Zirconium Oxide in a range of or substantially of 15% to 85%. Additionally, a composite ceramic material such as Zirconia toughed Alumina may be used. The ceramic materials used are all biocompatible. The ceramic acetabular cup 12 is formed as a unitary piece, and is hence described as being a monobloc ceramic acetabular cup 12. The monobloc ceramic acetabular cup 12 is devoid of a separate inner liner. The monobloc ceramic acetabular cup 12 comprises an internal concave surface which has a part-spherical surface. The part-spherical surface forms a first articulation surface 18. The first articulation surface 18 is preferably smooth to facilitate articulation. The monobloc ceramic acetabular cup 12 has a convex outer contact surface forming a bone-interfacing outer surface 20 for bone fixation when implanted into a patient.

In the depicted embodiment, the bone-interfacing outer surface 20 is part-spherical. A central point of the acetabulum defines a polar axis PA. A pole P is located on the polar axis PA. The part-spherical portion extends from the pole P of the monobloc ceramic acetabular cup 12 to a rim 22. The bone-interfacing outer surface 20 may include a flat or substantially flat portion adjacent to the monobloc ceramic acetabular cup rim 22. The flat or substantially flat portion may form a flared portion adjacent to the monobloc ceramic acetabular cup rim 22. However, it is envisaged that the flat portion may not be present. A multi-geometric outer surface may aid manufacture, implantation and/or fixation. It is envisaged that other different geometric outer surface profiles may be utilised.

The monobloc ceramic acetabular cup rim 22 preferably has an arcuate inner edge 24 which extends to meet the first articulation surface 18. The monobloc ceramic acetabular cup rim 22 also comprises an arcuate outer edge 26. The arcuate inner and outer edges 24, 26 are preferably uniform around a circumference of the monobloc ceramic acetabular cup rim 22. The arcuate of the inner and outer edges 24, 26 may be the same, substantially the same or different to one another. The upper most portion of the monobloc ceramic acetabular cup rim 22 is coincident with a first datum plane D1.

The radius of the first articulation surface defines a first active arc, which extends from the pole P to the arcuate inner edge of the monobloc ceramic acetabular cup rim 22. The active arc may extend between 150 and 220 degrees. Preferably, the active arc extends 160 to 200 degrees. Most preferably, the active arc extends 163 degrees.

A wall thickness of the monobloc ceramic acetabular cup 12, between the first articulation surface 18 and the bone-interfacing outer surface 20 is non-uniform. In the depicted embodiment, the wall thickness decreases in thickness towards the pole P. The change in wall thickness may be tapered. It is envisaged that at least a majority of the wall thickness may be uniform. Differences in wall thickness may be beneficial for assisting the introduction and/or removal of the ceramic acetabular cup during surgery.

The polyethylene articulation element 14 has a convex outer surface. The convex outer surface forms a second articulation surface 28. The polyethylene articulation element 14 is dimensioned to fit non-captively into the monobloc ceramic acetabular cup 12. The shape and dimension of the second articulation surface 28 is complementary to the shape and dimension of the first articulation surface 18. The first articulation surface 18 is rotatably engagable with the second articulation surface 28 of the polyethylene articulation element 14, in use.

The polyethylene articulation element 14 has a concave internal surface having a part-spherical portion which forms a third articulation surface 30, and a tapered portion 32. The radius of the third articulation surface 30 defines a second active arc, which extends to the tapered portion 32. The active arc extends to preferably less than 230 degrees.

The part-spherical portion 30 extends from the pole P. The tapered portion 32 in the depicted embodiment is frusto-conical. The tapered portion 32 extends from the termination of the part-spherical portion 30 to a rim 34 of the polyethylene articulation element 14. The upper most portion of the polyethylene articulation element rim 34 is coincident with a second datum plane D2. The tapered portion 32 has a cone angle of between 40 and 90 degrees relative to the second datum plane D2. Preferably the tapered portion 32 has a cone angle of 60 degrees relative to the second datum plane D2. The tapered portion 32 in use aids to limit the dislocation of the femoral head, and/or impact wear or damage on the head or neck of the femoral component.

The polyethylene articulation element rim 34 has an arcuate outer edge 36. The arcuate outer edge 36 extends to meet the second articulation element 28. The arcuate of the outer edge 36 is uniform and constant around the circumference of the polyethylene articulation element rim 34.

The polyethylene articulation element rim 34 includes a flat portion, which is positioned between the arcuate outer edge 36 and tapered portion 32. However, it is envisaged that the flat portion may not be present, and the arcuate outer edge 36 and the tapered portion 32 may extend to meet one another.

The polyethylene articulation element 14 is preferably made of a crosslinked polyethylene and/or a vitamin-E crosslinked polyethylene.

As with the monobloc ceramic acetabular cup 12, a wall thickness of the polyethylene articulation element 14 between the second articulation surface 28 and the third articulation surface 30 is non-uniform. In the depicted embodiment, the wall thickness decreases in thickness towards the pole P. Alternatively, it is envisaged the wall thickness between the second articulation surface 28 and the third articulation surface 30 may increase in thickness towards the pole P.

The ceramic femoral ball head 16 is made solely of ceramic. The ceramic femoral ball head 16 comprises a convex outer surface 38. The outer surface has a part-spherical portion, defines a fourth articulation surface 40. In selected embodiments, the outer surface 38 may have a tapered portion 42. The part-spherical portion 40. The fourth articulation surface 40 is shaped and dimensioned so as to be directly engageable against the third articulation surface 30 of the polyethylene articulation element 14. The fourth articulation surface 40 is dimensioned to allow for the ceramic femoral ball head 16 to be captively received by the polyethylene articulation element 14. The ceramic femoral ball head 16 preferably engages with the polyethylene articulation element 14 via push-fit engagement.

The outer surface tapered portion 42 extends from the end of the part-spherical portion 40, towards a rim 44 of the ceramic femoral ball head 16. The uppermost portion of the ceramic femoral ball head rim 44 is coincident with a third datum plane D3. The outer surface tapered portion 42 is frusto-conical in shape. The outer surface tapered portion 42 has a cone angle of 40 to 90 degrees relative to the third datum plane D3. Preferably the cone angle is 45 degrees relative to the third datum plane D3.

The ceramic femoral ball head 16 has a central bore 46. The central bore 46 is dimensioned to allow for attachment of an associated femoral stem. The central bore 46 is morse tapered to allow for friction fit engagement with the associated femoral stem. The central bore 46 has a frusto-conical end to restrict insertion of a femoral stem into the ceramic femoral ball head 16. An inlet 48 to the central bore 46 is tapered inwardly. The ceramic femoral ball head rim 44 is positioned between the outer surface tapered portion 42 and the inlet 48 to the central bore 46. The ceramic femoral ball head rim 44 includes a flat portion.

The first datum plane D1 is offset from the third and second datum planes D3, D2 to in-use reduce contact between the femur and monobloc ceramic acetabular cup 12. The second datum plane D2 is offset from the third datum plane D3 to reduce contact between the femur and polyethylene articulation element 14. The first articulation surface 18 has an equatorial centre point E1 on the polar axis PA. The second articulation 28 surface has an equatorial centre point E2 on the polar axis PA. When assembled, the equatorial centre point E1 of the first articulation surface 18 is offset from the equatorial centre point E2 of the third articulation surface 30. The offset may be in a positive direction away from the pole P.

As best illustrated in FIG. 3, the polyethylene articulation element 14 is inserted into the monobloc ceramic acetabular cup 12. Due to the dimension of the polyethylene articulation element 14, it is rotatable relative to the monobloc ceramic acetabular cup 12 along the first active arc. The ceramic femoral ball head 16 is inserted into the polyethylene articulation element 14 in a captive manner. The ceramic femoral ball head 16 is freely rotatable relative to the polyethylene articulation element 14 along the second active arc, and as such, relative to the monobloc ceramic acetabular cup 12. The fit of the ceramic femoral ball head 16 into the polyethylene articulation element 14 may be by an interference fit, a press fit or the like. Thus, whilst the polyethylene articulation element 14 retains the ceramic femoral ball head 16, the dimension of the polyethylene articulation element 14 and the smooth third and fourth articulation surfaces 30, 40 allow for rotational movement.

Similarly, the polyethylene articulation element 14 may also move relative to the monobloc ceramic acetabular cup 12, and/or the ceramic femoral ball head 16 about the smooth second articulation surface 28. The second articulation surface 28 being dimensioned to receptive and allow rotational movement along the first active arc.

FIG. 4a, FIG. 4b, FIG. 4c and FIG. 4d collectively show the dual mobility prosthesis 10 in use. A femoral stem 50 is inserted into the ceramic femoral ball head 16. FIG. 4a illustrates a first working condition of the dual mobility prosthesis 10, wherein the monobloc ceramic acetabular cup 12, polyethylene articulation element 14 and ceramic femoral ball head 16 are concentrically aligned. The polyethylene articulation element 14 is able to rotate freely relative to the ceramic femoral ball head 16 and to the monobloc ceramic acetabular cup 12. Likewise, the ceramic femoral ball head 16 is able to freely rotate relative to the polyethylene articulation element 14 and/or monobloc ceramic acetabular cup 12.

FIG. 4b illustrates a second working condition of the dual mobility prosthesis 10, wherein the ceramic femoral ball head 16 position is offset from the concentric alignment of the monobloc ceramic acetabular cup 12 and polyethylene articulation element 14. The ceramic femoral ball head 16 has rotated anti-clockwise relative to the polyethylene articulation element 14. As shown, the ceramic femoral ball head 16 can rotate freely with the fourth articulation surface 40 passing over the third articulation surface 30 along the second active arc. Although not depicted, the polyethylene articulation element 14 may also rotate freely with the second articulation surface 28 passing over the first articulation surface 18.

The rotation of the ceramic femoral ball head 16 may influence the rotation of the polyethylene articulation element 14. As shown in FIG. 4c, use, the ceramic femoral ball head 16 may rotate to such an extent relative to the polyethylene articulation element 14 that the femoral stem 50 contacts the tapered portion 32 of the polyethylene articulation element 14.

As shown in FIG. 4d, the contact of the femoral stem to the surface of the tapered portion 32 may in turn move the polyethylene articulation element 14 relative to the monobloc ceramic acetabular cup 12. The cone angle of the tapered portion 32 of the polyethylene articulation element 14 ensures the femoral stem is restricted from contacting the ceramic femoral ball head 16.

All of the articulation surfaces 18, 28, 30, 40 are preferably smooth and continuous. The first and second active arcs are offset from one another in use. The possibility of distinct and simultaneous rotation increases the range of motion that would be possible in use, when part of a total hip replacement.

FIG. 5 shows a system comprised of a plurality of monobloc ceramic acetabular cups 12, a plurality of polyethylene articulation elements 14 and a plurality of ceramic femoral ball heads 16.

As shown, at least one of the plurality of monobloc ceramic acetabular cups 12 is preferably of a different dimension and/or shape to at least one other monobloc ceramic acetabular cup 12. Conversely, at least one of the plurality of polyethylene articulation elements 14 is of a different dimension and/or shape to at least one other polyethylene articulation element 14. At least one of the plurality of ceramic femoral ball heads 16 is of a different dimension and/or shape to at least one other ceramic femoral ball head 16.

The arrows depict that the polyethylene articulation elements 14 and ceramic femoral ball heads 16 are able to articulate relative to one another and relative to the monobloc ceramic acetabular cups 12.

In use, ex vivo, a surgeon or surgical engineer may select a monobloc ceramic acetabular cup 12 from the plurality of monobloc ceramic acetabular cups 12 of a suitable size for a prepared acetabulum of a patient. The selection may be driven by the age, lifestyle of the patient. Alternatively, the condition of the hip acetabulum may drive the chosen monobloc ceramic acetabular cup 12.

The monobloc ceramic acetabular cup 12 may be chosen for the diameter of the bone-interfacing outer surface 20. It may be chosen for the wall thickness. It may be chosen for the first active arc of the first articulation surface 18. Alternatively, a surgeon or surgical engineer may choose a monobloc ceramic acetabular cup 12 with a specific wall thickness tapering. For example, a monobloc ceramic acetabular cup 12 with a thinner wall may be preferable to increase the range of motion possible for a patient. A monobloc ceramic acetabular cup 12 with an increased wall thickness near the monobloc ceramic acetabular cup rim 22 may be chosen for more active or younger patients, where increased protection against wear and tear may be important. The cup size may range from 46 mm to 70 mm.

Each monobloc ceramic acetabular cup 12 may have a different wall thickness. The choice of a monobloc ceramic acetabular cup 12 with a particular wall thickness may be directed by, at least in part, a lifestyle of the patient, and thus a typical range of hip motion required. A minimum wall thickness is determined by the required active arc. The larger the required active arc, the thinner the desired wall thickness. A greater offset datum plane allows for a larger active arc, resulting in a larger range of motion. A larger range of motion reduces the risk of impingement. However, a greater offset datum also increases the risk of the ceramic femoral ball head 16 being disengaged. Therefore, these factors will need to be balanced when choosing both a monobloc ceramic acetabular cup 12 and a polyethylene articulation element 14.

The surgeon may then select a ceramic femoral ball head 16 of an appropriate size. The selection may be influenced by the age of the patient. Preferable size of ceramic femoral ball head 16 includes 28 mm and 32 mm sized heads.

The surgeon may then select a polyethylene articulation element 14 having a second articulation surface 28 of a complementary dimension and shape to the first articulation surface 18. The second articulation surface 28 of the polyethylene articulation element 14 being of a complementary dimension and shape to the fourth articulation surface 40 of the ceramic femoral ball head 16. The complementary dimensions allowing for the polyethylene articulation element 14 to freely rotate relative to the monobloc ceramic acetabular cup 12 on the first and second articulation surfaces 18, 28. The complementary dimensions also allow for the ceramic femoral ball head 16 freely rotate relative to the monobloc ceramic acetabular cup 12 and/or polyethylene articulation element 14.

The polyethylene articulation element 14 selection may be driven by the wall thickness of the monobloc ceramic acetabular cup 12 and the radius of the ceramic femoral ball head 16. The polyethylene articulation element 14 selection may be driven by the active arc of the polyethylene articulation element 14 selection.

The polyethylene articulation element 14 and ceramic femoral ball head 16 may be selected as a head assembly engageable with the monobloc ceramic acetabular cup 12 and suitable for engagement with a femur of a patient. Selection of the head assembly may be drive by an articulation characteristic required by a patient.

Although the method of assembly of a dual mobility prosthesis has been described as ex vivo, this is optional, and the method of assembly of a dual mobility prosthesis may be performed, at least in part, in vivo.

Although a selection process made by the surgeon has been described, it is envisaged that the surgeon may choose the polyethylene articulation element 14 selection, ceramic femoral ball head 16 and monobloc ceramic acetabular cup 12 in any order deemed appropriate.

The surgeon may alternatively be provided with a set of polyethylene articulation element 14 and ceramic femoral ball head 16 and use them with commercially available acetabular cup. Additionally, the surgeon may alternatively be provided with a set of monobloc ceramic acetabular cups 12 and polyethylene articulation element 14 and use them alongside commercially available femoral ball heads. However, the use of femoral ball head and acetabular cups that are not formed from ceramic is envisaged to be disadvantageous.

In addition to the monobloc ceramic acetabular cups 12, polyethylene articulation elements 14 and ceramic femoral ball head 16, the surgeon may be provided with a stem suitable for insertion in a prepared femur.

A bone-fixation coating may be applied to the bone-interfacing outer surface 20 of the monobloc ceramic acetabular cup 12. Biocompatible metals and metal alloys are suitable for the bone-fixation coating. The bone-fixation coating may be for example, Titanium based. The use of a bone-fixation coating negates the need for a metal shell. The ceramic acetabular cup may directly contact the bone. In order to facilitate and accelerate ingrowth of the implant into the bone, the coating may include a bioactive component. A suitable component may be hydroxyapatite for example.

Although a bone-fixation coating has been described, it may be feasible to simply roughen the outer surface of the acetabular cup, to encourage bone fixation.

Although a wall thickness of the monobloc ceramic acetabular cup has been described as decreasing in thickness towards the pole, it is envisaged the wall thickness may increase in thickness towards the pole.

Although the articulation surfaces have been referred to as: a first articulation surface; a second articulation surface; a third articulation surface; and a fourth articulation surface, the terms may be interchanged for: cup articulation surface; an outer articulation surface; an inner articulation surface; and a head articulation surface respectively.

Although not described, the ceramic femoral ball head may also be monobloc.

Part-spherical portion of inner surface of the polyethylene articulation element and third articulation surface are interchangeable terms, as the part-spherical portion defines the third articulation surface.

Part-spherical portion of ceramic femoral ball head and fourth articulation surface are interchangeable terms, as the part-spherical portion defines the fourth articulation surface.

The present invention provides a dual mobility prosthesis with only ceramic and polyethylene components. Ceramic has excellent biocompatibility, high surface hardness and wear resistance. The use of a polyethylene articulation element acts as an articulating bearing providing range of motion benefits. The absence of metal in the monobloc ceramic acetabular cup, a polyethylene articulation element and a ceramic femoral ball head prevents carcinogenic and genotoxic effects. The present invention also negates the need for a liner, thus a thin-walled monobloc ceramic acetabular cup can be used, which allow for a large diameter polyethylene articulation element resulting in a larger range of articulation.

The words ‘comprises/comprising’ and the words ‘having/including’ when used herein with reference to the present invention are used to specify the presence of stated features, integers, steps, or components, but do not preclude the presence or addition of one or more other features, integers, steps, components, or groups thereof.

It is appreciated that certain features of the invention, which are, for clarity, described in the context of separate embodiments, may also be provided in combination in a single embodiment. Conversely, various features of the invention which are, for brevity, described in the context of a single embodiment, may also be provided separately or in any suitable sub-combination.

The embodiments described above are provided by way of examples only, and various other modifications will be apparent to persons skilled in the field without departing from the scope of the invention as defined herein.

1. A dual mobility prosthesis for implantation into an acetabulum, the dual mobility prosthesis comprising:
a monobloc ceramic acetabular cup having an internal concave surface forming a first articulation surface;
a polyethylene articulation element having a convex outer surface forming a second articulation surface, and a concave internal surface forming a third articulation surface, the second articulation surface being rotatably engageable with the first articulation surface of the monobloc ceramic acetabular cup; and
a ceramic femoral ball head having a convex outer surface forming a fourth articulation surface directly engageable against the third articulation surface of the polyethylene articulation element, the ceramic femoral ball head being attachable to a femoral stem;
wherein the first articulation surface is dimensioned to contact the second articulation surface and covers less than a hemisphere to non-captively receive the polyethylene articulation element, and wherein a wall thickness of the monobloc ceramic acetabular cup is non-uniform.

2. The dual mobility prosthesis as claimed in claim 1, wherein the third articulation surface is dimensioned to contact the fourth articulation surface and covers more than a hemisphere to captively receive the ceramic femoral ball head.

3. The dual mobility prosthesis as claimed in claim 1, wherein the ceramic femoral ball head connects to the polyethylene articulation element via a push-fit mechanism.

4. The dual mobility prosthesis as claimed in claim 1, wherein the polyethylene articulation element is made of one or more of: an Ultra High Molecular Weight Polyethylene, crosslinked polyethylene, or a vitamin-E crosslinked polyethylene.

5. The dual mobility prosthesis as claimed in claim 1, wherein the polyethylene articulation element directly engages with the monobloc ceramic acetabular cup without a separate liner.

6. The dual mobility prosthesis as claimed in claim 1, wherein an equatorial centre point of the first articulation surface is offset from an equatorial centre point of the third articulation surface.

7. The dual mobility prosthesis as claimed in claim 1, wherein the monobloc ceramic acetabular cup has a convex outer contact surface engageable with an acetabulum of a patient.

8. The dual mobility prosthesis as claimed in claim 7, wherein the convex outer contact surface flares at or adjacent to a rim of the monobloc ceramic acetabular cup.

9. The dual mobility prosthesis as claimed in claim 1, wherein the second articulation surface has an articulation angle of at least 10 degrees greater than an articulation angle of the first articulation surface.

10. The dual mobility prosthesis as claimed in claim 1, wherein the fourth articulation surface has an articulation angle of at least 10 degrees greater than an articulation angle of the third articulation surface.

11. The dual mobility prosthesis as claimed in claim 1, wherein the concave internal surface of the polyethylene articulation element has a tapered portion.

12. The dual mobility prosthesis as claimed in claim 11, wherein the tapered portion is a frusto-conical portion.

13. The dual mobility prosthesis as claimed in claim 12, wherein the frusto-conical portion of has a cone angle of between 40 degrees and 90 degrees.

14. The dual mobility prosthesis as claimed in claim 12, wherein the tapered portion extends from or is adjacent to a rim of the polyethylene articulation element.

15. The dual mobility prosthesis as claimed in claim 1, wherein the wall thickness decreases towards a rim of the monobloc ceramic acetabular cup.

16. The dual mobility prosthesis as claimed in claim 1, wherein the wall thickness increases towards a rim of the polyethylene articulation element.

17. A dual mobility prosthesis system comprising:
a plurality of monobloc ceramic acetabular cups, each monobloc ceramic acetabular cup having a cup articulation surface;
a plurality of polyethylene articulation elements, each polyethylene articulation element having an inner articulation surface, and an outer articulation surface, the outer articulation surface being rotatably engageable with the cup articulation surface of the acetabular cup; and
a plurality of ceramic femoral ball heads, each ceramic femoral ball head having a head articulation surface directly engageable against the inner articulation surface of the polyethylene articulation element, the ceramic femoral ball head being attachable to a femoral stem;
wherein the cup articulation surface of each monobloc ceramic acetabular cup is dimensioned to contact the inner articulation surface of a selected polyethylene articulation element and covers less than a hemisphere to non-captively receive the selected polyethylene articulation element, and wherein a wall thickness of the monobloc ceramic acetabular cup is non-uniform.

18. The dual mobility prosthesis system as claimed in claim 17, wherein one or more of:
at least one of the plurality of monobloc ceramic acetabular cups is one or both of: a different dimension or a different shape to at least one other monobloc ceramic acetabular cup,
at least one of the plurality of polyethylene articulation elements is one or both of: a different dimension or a different shape to at least one other polyethylene articulation element, or
at least one of the plurality of ceramic femoral ball head is one or both of: a different dimension or a different shape to at least one other ceramic femoral ball head.

19.-22. (canceled)
